(19) United States
(12) Patent Application Publication
(10) Pub. No.: US 2024/0399036 A1
(43) Pub. Date: Dec. 5, 2024

(54) DEVICE FOR MONITORING BLOOD PURIFICATION USING AN EXTRACORPOREAL BLOOD PURIFICATION DEVICE

(71) Applicant: FRESENIUS MEDICAL CARE DEUTSCHLAND GMBH, Bad Homburg (DE)

(72) Inventor: Andreas MAIERHOFER, Wiesbaden (DE)

(73) Assignee: FRESENIUS MEDICAL CARE DEUTSCHLAND GMBH, Bad Homburg (DE)

(21) Appl. No.: 18/700,276

(22) PCT Filed: Oct. 11, 2022
(86) PCT No.: PCT/EP2022/078320

§ 371 Date: Apr. 11, 2024

(30) Foreign Application Priority Data
Oct. 14, 2021 (DE) 10 2021 126 681.5

(51) Int. Cl. A61M 1/16; A61M 1/34; G01N 27/06; G01N 33/487

(52) U.S. Cl. CPC A61M 1/1605; A61M 1/3406; A61M 1/3413; G01N 27/06; G01N 33/48707; A61M 2205/502; A61M 2205/52; A61M 2205/581

(57) ABSTRACT

The invention relates to a method and a device for monitoring blood purification with an extracorporeal blood purification device which is designed such that a blood purification unit 1 is used to perform blood purification with predetermined treatment parameters Q_{b} in an extracorporeal blood circuit 9. The concentration of a substance is measured during the blood purification with at least one sensor 31, 32, 33, 34 and a parameter K which is characteristic of the purifying performance of the blood purification unit 1 is determined with a computing and/or evaluation unit 25 on the basis of the measured concentration of a substance. The parameter K which is characteristic of the purifying performance of the blood purification unit 1 is compared with an expected value K_{ref}. To this end, a tolerance range is determined for the expected value using the computing and/or evaluation unit 25, wherein actions that are predetermined by the computing and/or evaluation unit 25 are triggered depending on whether the parameter which is characteristic of the purifying performance of the blood purification unit lies within or outside the tolerance range for the expected value.

The invention relates to a method for monitoring blood purification with an extracorporeal blood purification device and to a device for monitoring blood purification for use with an extracorporeal blood purification device, wherein the blood purification device is designed such that a blood purification unit is used to perform blood purification with predetermined treatment parameters in an extracorporeal blood circuit. The invention also relates to an extracorporeal blood purification device with a device for monitoring blood purification and to a blood purification system comprising at least two extracorporeal blood purification devices and a data processing system.

The main tasks of extracorporeal blood purification methods include the removal of substances excreted in the urine, e.g. urea, beta-2-microglobulin, phosphate and other uraemic toxins, from the blood, as well as the supply of certain substances, e.g. bicarbonate. For this purpose, an exchange of substances takes place in a blood purification unit (dialyser, filter, adsorber), which causes the substance concentration to change from the blood-side inlet to the outlet as the blood flows through. The measure of the quality of the exchange is the substance-specific clearance K, which describes the proportion of the blood which has been completely purified of the substance in question:

$$K = {Q}_{b} \frac{{c}_{bi} -}{{c}_{bi}} \frac{{c}_{bo}}{} \qquad Equation ( 1 )$$

c_{bi}: Substance concentration in the blood at the inlet of the dialyser's blood chamber
c_{bo}: Substance concentration in the blood at the outlet of the dialyser's blood chamber
Q_{b} Blood flow

Various types of blood purification devices are known. During the blood purification, the patient's blood flows in an extracorporeal blood circuit through the blood purification unit. The known blood purification devices include, for example, the devices for haemodialysis, haemofiltration and haemodiafiltration. In these devices, the blood purification unit is a dialyser or filter which is separated into a first and second compartment by a semipermeable membrane. During haemodialysis, the blood flows through the first compartment (blood chamber) of the dialyser, which is part of an extracorporeal blood circuit, while the dialysis fluid flows through the second compartment (dialysis fluid chamber) of the dialyser, which is part of a dialysis fluid system. During apheresis, blood components or substances are removed from the blood with a blood purification unit (cell separator) in an extracorporeal blood circuit.

If the blood purification is carried out with a dialyser by the exchange of substances with a dialysis fluid via a semipermeable membrane, the dialysance D can be defined instead of the clearance K if the substance of interest is also present on the dialysate side:

$$D = {Q}_{b} \frac{{c}_{bi} -}{{c}_{bi} -} \frac{{c}_{bo}}{{c}_{di}} \qquad Equation ( 2 )$$

c_{bi}: Substance concentration in the blood at the inlet of the dialyser's blood chamber
c_{bo}: Substance concentration in the blood at the outlet of the dialyser's blood chamber
c_{di}: Substance concentration in the dialysis fluid at the inlet of the dialyser's dialysis fluid chamber
Q_{b}: Blood flow

The clearance K and dialysance D are substance-dependent variables and ideally only depend on the characteristics of the “artificial kidney” and the predetermined treatment parameters that the user can set on the blood purification device. This includes above all the extracorporeal blood flow Q_{b}. In haemodialysis, furthermore, the dialysis fluid flow Q_{d} is relevant. In convective methods (haemo(dia)filtration), the convective flow of the substitution solution Qₛ is a further treatment parameter.

In practice, however, K and D deviate from the values expected under ideal conditions. The main cause is that the characteristics of the blood purification are typically determined under laboratory conditions, whereby not all parameters relevant in clinical use, for example patient-specific properties of the blood and the extracorporeal blood circuit or flow conditions in the dialyser that have changed due to blood properties, can be taken into account. Furthermore, on the device side, in the blood purification device there may be undetected deviations between the user's specified values and the actually existing flow conditions.

In principle, K or D can be determined by determining the concentrations according to equation (1) and equation (2). Blood-side measurements that require a sensor to come into direct contact with the blood, however, involve the risk of contamination, and therefore they are generally not used for the online determination of K or D. Most of the methods therefore use sensors on the dialysate side, which are based on the assumption that the mass balance is conserved across the semipermeable membrane. This assumption does not apply, or applies only to a limited extent, when using absorbers or membranes with absorber properties.

A known method for online determination of K or D is based on the dialysate-side determination of the sodium dialysance by generating a temporary variation in the sodium content upstream of the dialyser and measuring the dialysate-side response of the system downstream. The variation is preferably quantified here by measuring a variable that correlates with the sodium concentration or the variation in the sodium concentration, in particular the conductivity of the dialysis fluid. The sodium dialysance determined in this way (in ml/min) is then equated with the urea clearance. There are also models for other substances (e.g. potassium, creatinine, bicarbonate, etc.) that determine the substance-specific clearance K or dialysance D by multiplying the sodium dialysance by a substance-specific proportionality factor. By integrating the continuously determined clearance K, if the distribution volume V is known, the dialysis dose Kt/V or Dt/V can be determined.

Other known methods are based on the selective measurement of a substance concentration in the dialysate downstream of the dialyser, or a variable that correlates with this substance concentration (e.g. spectral absorption in the UV range or visible range of light). These methods are based on the assumption that the change in the substance concentration on the blood side is proportional to the change on the dialysate side. The dialysis dose (KtV)j or (DtV)j in a time interval tj is determined from the change over time of the dialysate-side signal, assuming an exponential drop (single-pool model). The total dialysis dose then results from the sum of the dialysis doses in the time intervals. The mean clearance Kj in the time intervals tj can then also be derived from the knowledge of the distribution volume V.

A method and a device for determining the clearance K or dialysance D during extracorporeal blood purification, which is based on the online measurement of the electrolyte transfer at two different dialysate concentrations, are known for example from DE 39 38 662 A1 (U.S. Pat. No. 5,100,554) and DE 197 47 360 A1 (U.S. Pat. No. 6,156,002).

Methods for determining K or D generally require an accurate measurement of the blood-side and dialysate-side flows at the blood purification device. Errors in the determination of these flows therefore have a direct effect on the values of K and D. Errors in the determination of the dialysate flow Q_{d} can arise, for example, from incomplete filling of the balancing chambers used for balancing the dialysate-side flow. However, measurement with flow sensors (e.g. Coriolis flow meters) can also be error-prone due to a malfunction of the sensor. Deviations in the blood flow actually conveyed on the blood side from the target value depend on the type of conveyance. When using peristaltic pumps, deviations arise due to the reduction in the flow due to negative pressure on the suction side as well as due to changing elastic properties of the compressed hose segment. An occlusion at the inlet of the dialyser on the positive pressure side can also lead to an undetected decrease in the blood flow. When using impeller pumps, the blood flow depends to a large extent on the viscous properties of the medium and the flow resistance of the system, especially on the dialyser, and therefore no reliable delivery is possible without precise blood-side flow sensors. In addition to the absolute flow rate, the relative direction of blood to dialysate flow in the dialyser is also important for the value of K or D, since a greater exchange of substances is achieved when connected in countercurrent than when connected in cocurrent. Since the connection on the blood and dialysate side usually takes place manually, this can lead to unintentional mix-ups.

Pumps similar to those used for conveying blood are used to convey a substitution fluid during haemofiltration or haemodiafiltration. There are also methods in which the convective transmembrane flow is established solely through the pressure difference between the blood and dialysate side. Since the pressure conditions along the dialyser fibres change and can also reverse in the longitudinal course, the resulting net current for a certain substance can a priori be estimated only very imprecisely.

In addition, it must be taken into account that the purifying performance also depends on the mixing and flow conditions in the patient, especially in the vascular access. Recirculation, which can take place both directly between the arterial and venous connection point and systemically as so-called cardiopulmonary recirculation, leads to blood that has been purified in the blood purification device and returned venously into the circulation arriving at the arterial extraction point again without first having been mixed with the relevant solution volume throughout the body. This reduces the effective purifying performance of the blood purification unit. This occurs in particular when the flow of blood in the vascular access of the patient, the so-called shunt flow Qα, is smaller than the extracorporeal blood flow Q_{b}. Both the pulsatile nature of the shunt flow Qα, caused by the heartbeat, as well as the conveying process, in particular in peristaltic pumps, can lead to direct recirculation, even if Qα>Q_{b} on average. Unfavourable geometric arrangements of the cannulas (needles) relative to one another, e.g. cannulas that are arranged too close to one another, can also lead to direct recirculation. In contrast with the unavoidable cardiopulmonary recirculation, the latter is an effect that needs to be detected and avoided.

What is known as the dialysis dose Kt/V is of critical importance for the effectiveness of a dialysis treatment, and is defined as the quotient of the product of the clearance K for urea and effective treatment time t of the dialysis treatment and the distribution volume V of the patient for urea. For quality control, in practice the dialysis dose Kt/V (or Dt/V) achieved at the end of the dialysis treatment is compared with a minimum value. However, this comparison with an absolute value does not generally allow the early detection of more subtle problems in the treatment, which only lead to small deviations of Kt/V. In addition, the methods and devices used for the online determination of K and D are influenced by various sources of error.

The invention is based on the object of specifying a method that allows reliable monitoring of the operating state of a blood purification device, in particular specifying a method by means of which deviations from the normal operating state of a blood purification device can be detected at an early stage. Furthermore, the invention is based on the object of providing a device for monitoring blood purification for use with an extracorporeal blood purification device, by means of which the blood purification can be reliably monitored. Another object of the invention is to provide a blood purification system that allows reliable monitoring of blood purification.

These objects are achieved with the features of the independent claims. The dependent claims relate to preferred embodiments of the invention.

The method according to the invention allows for the monitoring of blood purification with an extracorporeal blood purification device which is designed such that blood purification is carried out with predetermined treatment parameters by means of a blood purification unit in an extracorporeal blood circuit.

The blood purification device can be any device suitable for performing haemodialysis, haemofiltration, haemo (dia) filtration, or a combination of these blood purification methods. Such blood purification devices belong to the prior art. With these blood purification devices, blood is taken from the patient from a vascular access via an (arterial) cannula (needle) and supplied to a blood purification unit via a blood line. The delivery pump for taking blood can be a part of the blood purification device or integrated into a disposable intended for single use, wherein any method suitable for conveying the blood, in particular by means of peristaltic pumps or impeller pumps, can be used. In the case of haemodialysis, haemofiltration and haemo (dia) filtration, the blood purification device can have means for dialysate preparation and supply lines or drains to the blood purification unit as well as means for withdrawing fluid by means of ultrafiltration.

According to the method according to the invention, the concentration of a substance or a variable correlating with the concentration of a substance is measured with at least one sensor during the blood purification and, on the basis of the concentration of a substance measured with the at least one sensor or a variable correlating with the concentration of a substance, at least one parameter which is characteristic of the purifying performance of the blood purification unit during the blood purification performed with the predetermined treatment parameters is determined using a computing and/or evaluation unit. Such methods for determining a variable which is characteristic of the blood purification, in particular the clearance K or dialysance D, are known.

To determine the variable which is characteristic of the blood purification, in particular the clearance K or dialysance D, means can be used to change the composition of the dialysis fluid by changing the mixing ratio of components involved in the online generation of the dialysis fluid. The concentration of a substance can be increased as well as decreased. The components of the dialysis fluid can also be provided in containers, for example bags, with a liquid or a solid being added from a separate reservoir for the measuring process, as a result of which the composition of the dialysis fluid is changed.

The measurement of the concentration of a substance or a variable correlating with the concentration of a substance can take place during the blood purification with blood-side sensors upstream and/or downstream of the blood purification unit and/or with dialysate-side sensors upstream and/or downstream of the blood purification unit, which sensors are suitable for determining the concentration of a substance contained in the blood or in the dialysate or a variable correlating therewith by any method using a contact-based or contactless measurement. In particular, these sensors can be ion-selective electrodes, conductivity sensors, spectroscopic devices for measuring in the infrared or visible range of light or in the UV range of light, sensors used in chromatographic methods (e.g. capillary electrophoresis), or amperometric sensors (e.g. glucose sensor). The connection of the sensors to the measuring points on the blood and/or dialysate side can be permanently set up in the blood purification device. However, the sensors can also be inserted only when the blood purification device is installed. They can also be a part of a disposable on the blood and/or dialysate side. The connection of the sensors can be wired or wireless.

A computing and/or evaluation unit is to be understood as any device which is suitable for using any method to determine a variable which is characteristic of the purifying performance of the blood purification unit from the measured value or the measured values. The method according to the invention is characterised in that not only is a variable which is characteristic of the purifying performance determined with the computing and/or evaluation unit, but also in that the computing and/or evaluation unit also determines an expected value for a parameter which characterises the purifying performance of the purification unit, which expected value is dependent on at least one treatment parameter.

The expected value can be determined by many different methods. The expected value can be a global value or a value that is specific to the treatment parameters of a patient which are set on the dialysis machine, a certain substance or a substance class, a time of day or season, a dialysis centre or a combination of various parameters. The expected value can be a value averaged over an intradialytic time interval.

The expected value can be calculated on the basis of a mathematical model or can be determined by accessing tabulated values. Furthermore, the expected value can be established by comparison with other treatments. These can be treatments with the same or similar treatment parameters or with treatment parameters that differ from the present treatment, from which the expected value for the treatment parameters of the current treatment is then determined on the basis of a mathematical model. These can be current treatments from the same medical centre or other centres, or historical treatments of the patient currently being treated on the device, or a combination of current and historical values.

According to the method according to the invention, the variable characteristic of the purifying performance of the blood purification unit is compared with the expected value. To this end, a tolerance range is determined for the expected value using the computing and/or evaluation unit, wherein actions that are predetermined by the computing and/or evaluation unit are triggered depending on whether the parameter which is characteristic of the purifying performance of the blood purification unit lies within or outside the tolerance range for the expected value. The comparison of the variable which is characteristic of the purifying performance of the blood purification unit, which can be measured during the blood purification, with the expected value allows for the continuous detection of deviations in the operating state of the blood purification device and the system consisting of the blood purification device and the patient from a typical or ideal operating state. The user can be advised of intolerable deviations and/or requested to initiate suitable measures. In the event of intolerable deviations, suitable measures can also be carried out automatically.

In contrast with the known monitoring of an absolute value of a variable which is characteristic of the purifying performance of the blood purification unit within the scope of quality control, a relative variable is determined for the predetermined treatment parameter or parameters that relates to a typical or ideal blood purification. The computing and/or evaluation unit can perform different actions depending on whether or not a deviation can be tolerated.

In the event of intolerable deviations, graphical elements and/or symbols can be displayed with a graphical user interface and/or acoustic signals can be generated with an acoustic user interface, which interfaces display to the user that the parameter which is characteristic of the purifying performance of the blood purification unit lies within or outside the tolerance range for the expected value. The user interface can be, for example, a screen, in particular a touchscreen, on which the graphical elements and/or symbols are displayed. The graphical elements can be, for example, dots, dashed lines, bars or areas that indicate the parameter which is characteristic of the purifying performance of the blood purification unit as a function of the treatment time, the upper and/or lower limits of the tolerance range, a deviation from the tolerance range or an exceedance of the tolerance range. The symbols can have a meaning that prompts the user to take certain measures. An acoustic user interface can emit an acoustic alarm in the event of an intolerable deviation.

If the tolerance range is exceeded, the user interface can also offer help in finding the cause. For the purpose of building up a knowledge base (“assisted machine learning”), causes identified by the user can also be input, for example. This can be done in free text or by selecting suggested causes. These user annotations, together with the relevant treatment and technical parameters, can then be sent to a server or a cloud for further processing.

For the automated initiation of selected actions, the control and/or evaluation unit can generate an electrical signal which signals that the parameter which is characteristic of the purifying performance of the blood purification unit is within or outside the tolerance range for the expected value. This electrical signal can be processed further in the device for monitoring the blood purification device or in a device interacting with the monitoring device, in particular the blood purification device.

One embodiment of the method according to the invention provides that expected values for different treatment parameters are stored in a memory, wherein the relevant expected value for the predetermined treatment parameters is read out from the memory by the computing and/or evaluation unit. The expected values can be stored in the memory, for example in the form of a table.

In a preferred embodiment, and knowing the properties of the blood purification unit used, the computing and/or evaluation unit is used to calculate the expected value according to a mathematical model which describes the expected value as a function of the predetermined treatment parameters. A predetermined treatment parameter is the blood flow. The calculation of the expected value can take into account whether postdilution or predilution takes place. The properties of the purification unit can be determined by laboratory measurements (manufacturer information). In this preferred embodiment, a “measured value” for a parameter describing the purifying performance of a blood purification unit is thus compared with a “calculated value”. For the calculation of the expected value according to the known models, measurements of changes in concentration in the extracorporeal blood circuit or dialysis fluid system are not required.

In a further embodiment, for determining the expected value, a parameter which is characteristic of the purifying performance of the blood purification unit is determined during blood purification with a different extracorporeal blood purification device than the one to be monitored and is read into a memory. The parameter which is characteristic of the purifying performance of the blood purification unit can be measured using the known methods. The expected value determined during a prior blood purification with the different extracorporeal blood purification device is then read out from the memory as the expected value for the blood purification with the blood purification device to be monitored. This embodiment assumes that at least two blood purification devices interact with one another, with an exchange of data taking place between the blood purification devices.

In the method according to the invention, the computing and evaluation unit can be a computing and/or evaluation unit which is spatially separated from the blood purification device and/or the memory can be a memory which is spatially separated from the blood purification device. The expected value can thus be determined on an external device in a medical centre or outside the medical centre by means of cloud computing. The transmission of raw data and/or calculated values from the device for monitoring blood purification to external computing units (cloud applications) or the transmission of expected values from external computing units to the device for monitoring the blood purification can take place via a data interface.

The device according to the invention for monitoring blood purification is designed to carry out the method according to the invention. The monitoring device according to the invention can form an external unit which records measured values by means of sensors, or it can be a component of the extracorporeal blood purification device.

The blood purification system according to the invention comprises at least two extracorporeal blood purification devices which are each designed such that blood purification is carried out with predetermined treatment parameters by means of a blood purification unit in an extracorporeal blood circuit, wherein the blood purification devices each have a data interface. The blood purification devices each have at least one sensor for determining the concentration of a substance or a variable correlating with the concentration of a substance during the blood purification and a computing and/or evaluation unit which is configured such that, on the basis of the concentration of a substance measured with the at least one sensor or a variable correlating with the concentration of a substance, at least one parameter is determined which is characteristic of the purifying performance of the blood purification unit during the blood purification performed with the predetermined treatment parameters. In this embodiment, the monitoring device is part of the blood purification device.

Furthermore, the blood purification system comprises a data processing system with which the at least two blood purification devices interact such that data are exchanged via the data interface between the at least two blood purification devices on the one hand and/or between at least one of the blood purification devices and the data processing system, on the other hand.

Furthermore, the blood purification system comprises a computing and/or evaluation unit which is configured such that a parameter which is characteristic of the purifying performance of the blood purification unit, which parameter is determined during a blood purification with one of the at least two blood purification devices, is read into a memory and is read out from the memory by another blood purification device of the at least two blood purification devices as the expected value. The computing and evaluation unit can be formed by components of the at least two blood purification devices or components of the data processing system. The memory can be part of the central data processing unit and/or the blood purification devices. Communication between the individual devices can take place via the Internet (cloud computing).

Embodiments of the invention will be explained in greater detail below with reference to the figures.

In the figures:

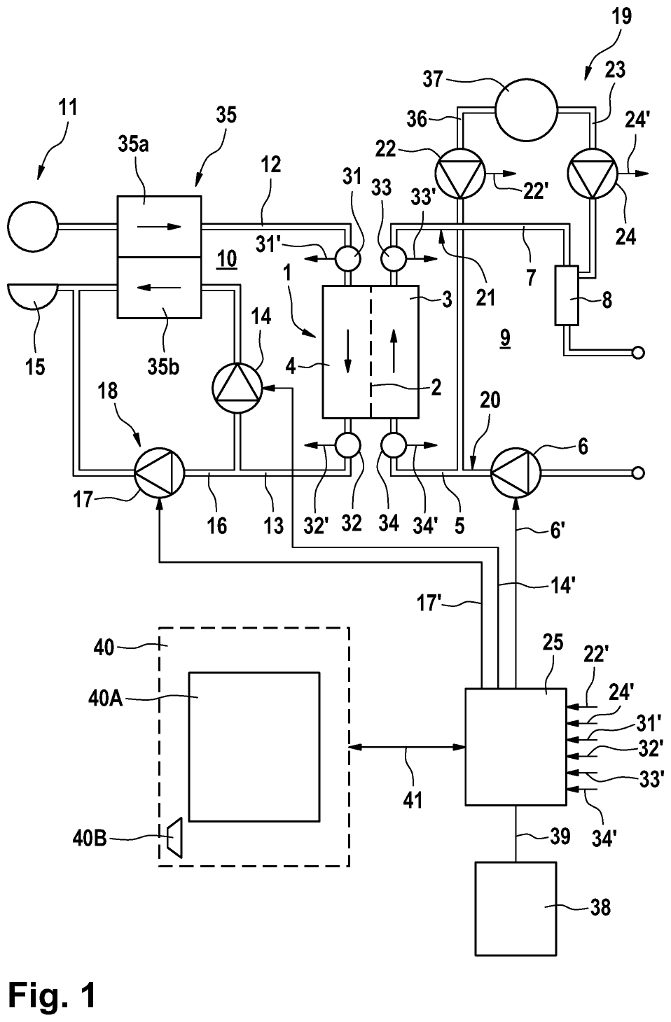

FIG. 1 shows a simplified schematic view of the essential components of an extracorporeal blood purification device according to the invention,

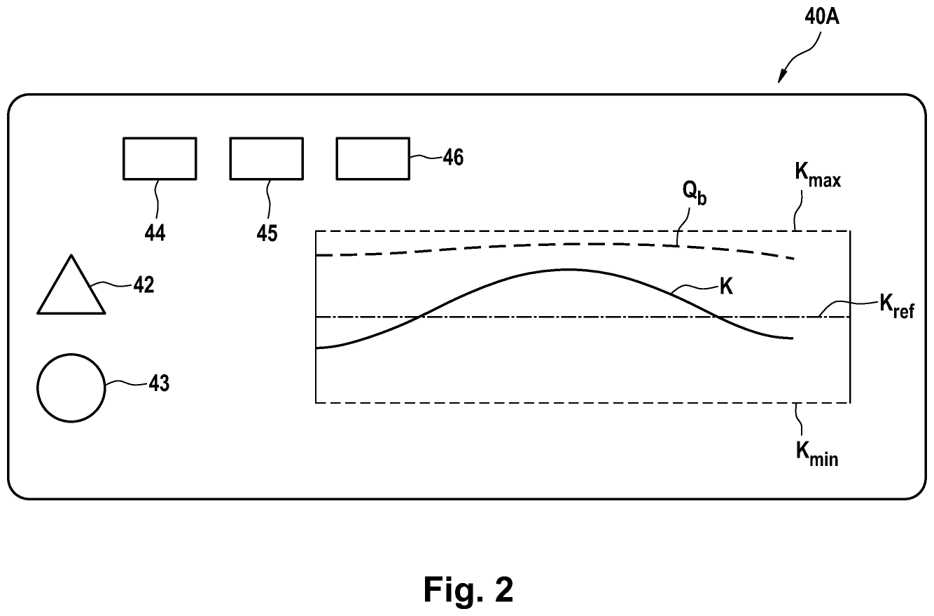

FIG. 2 shows the screen of the blood purification device with the measured clearance within the tolerance range,

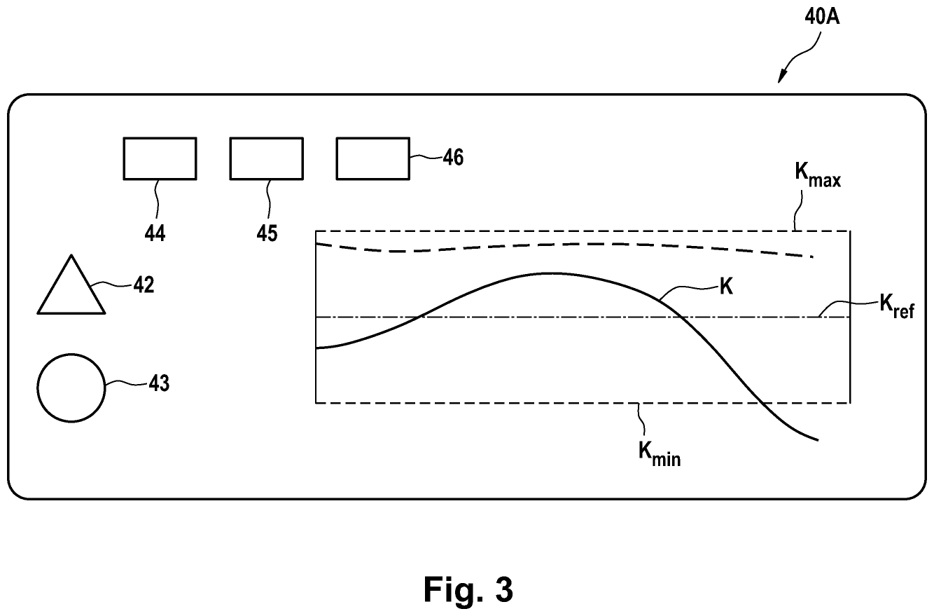

FIG. 3 shows the screen of the blood purification device with the measured clearance outside the tolerance range,

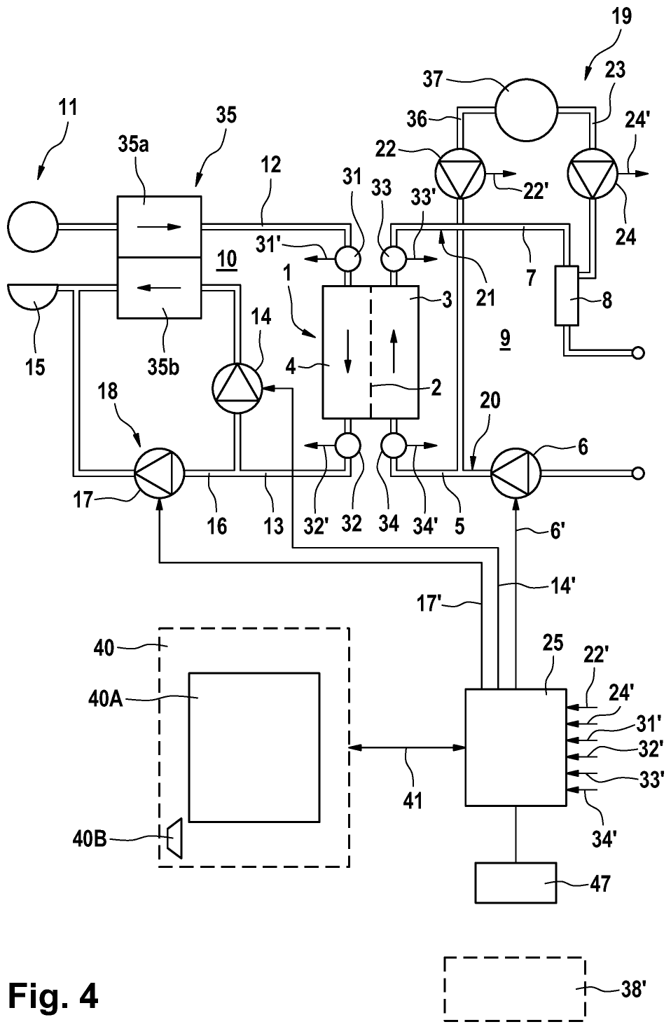

FIG. 4 shows a simplified schematic view of the essential components of an alternative embodiment of the extracorporeal blood purification device according to the invention,

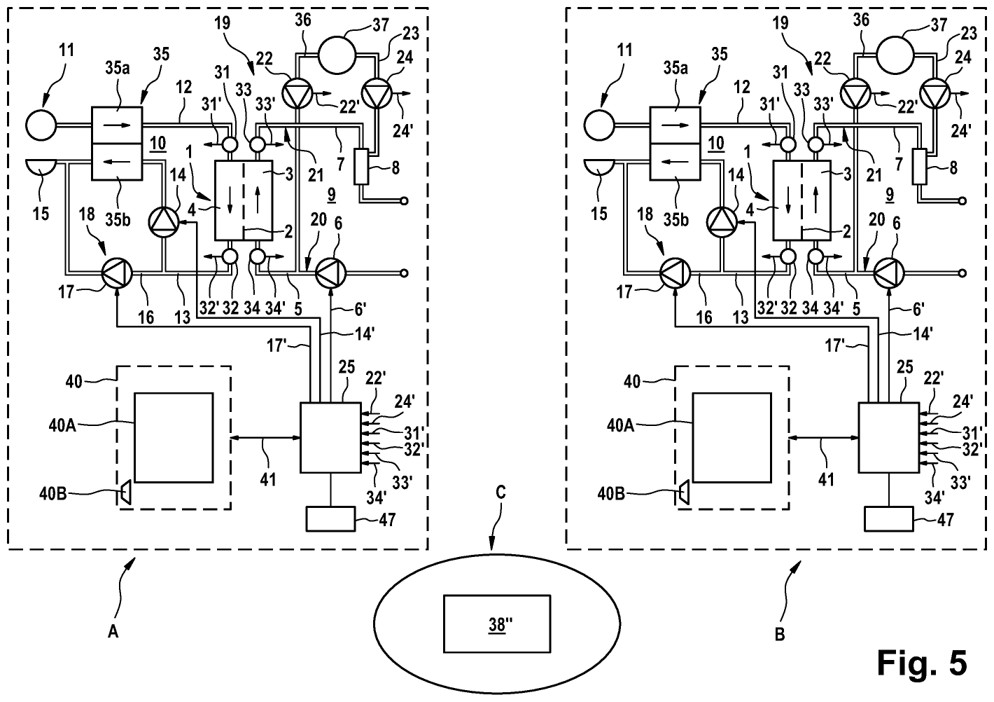

FIG. 5 shows a blood treatment system comprising two blood purification devices and a data processing system,

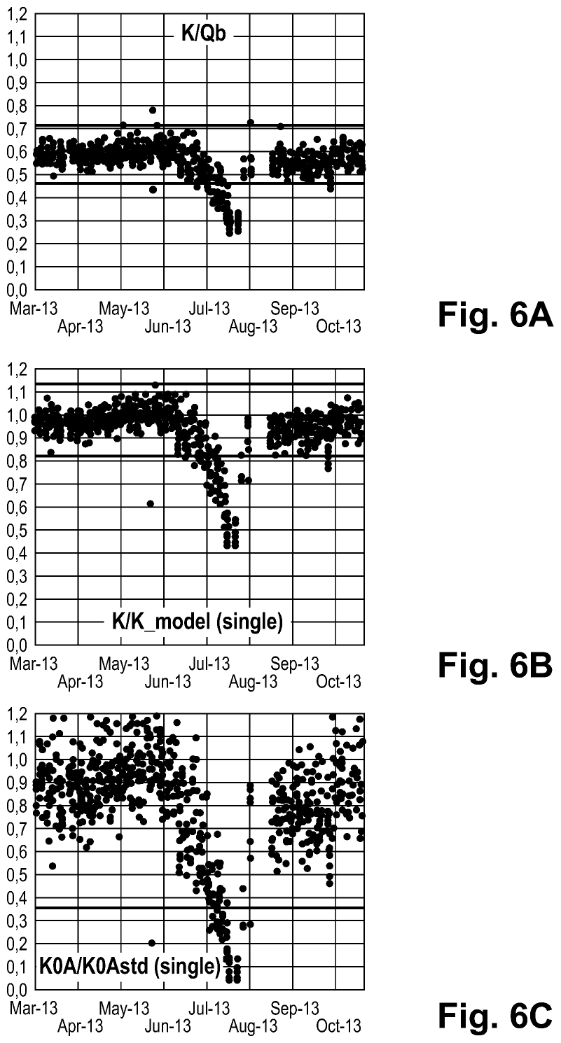

FIG. 6A to 6C show a trend analysis based on all individual clearance measurements,

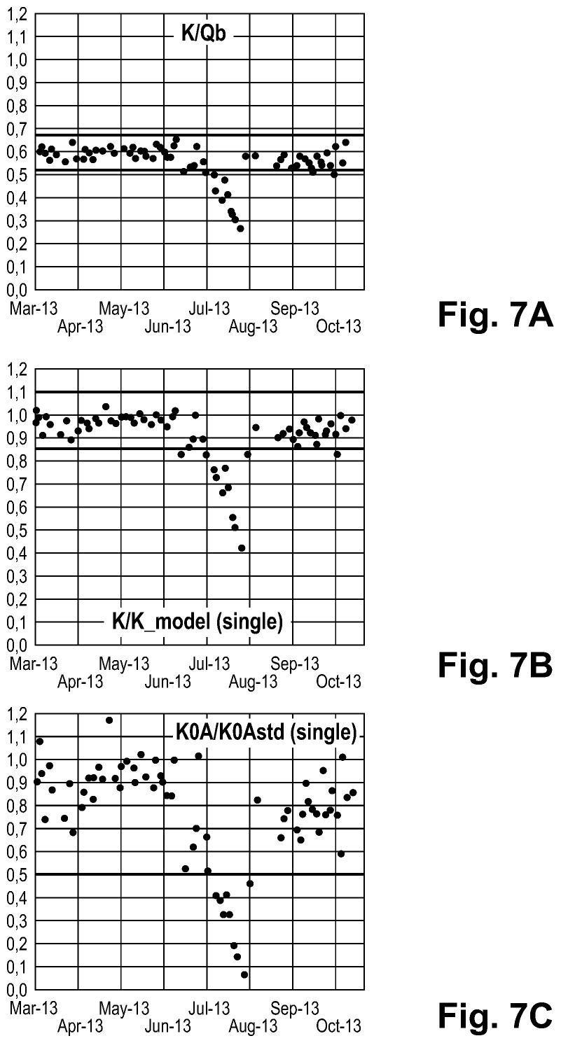

FIG. 7A to 7C show a trend analysis based on individual clearance measurements at selected times,

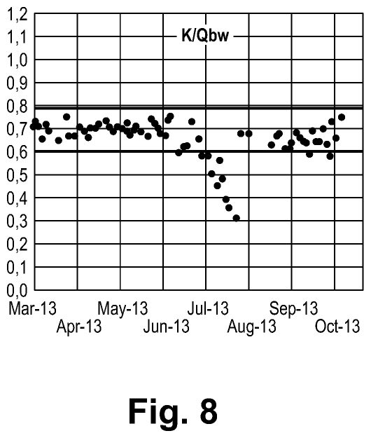

FIG. 8 shows a trend analysis based on individual clearance measurements at selected times, with the blood water flow as the reference,

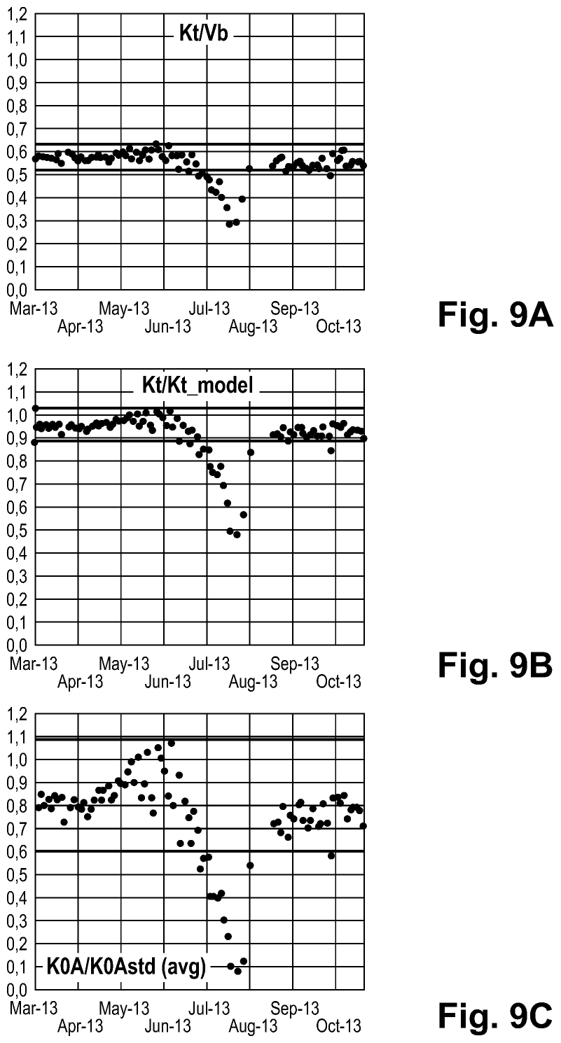

FIG. 9A to 9C show a trend analysis based on the total dialysis dose of the respective treatments.

FIG. 1 shows an embodiment of an extracorporeal blood purification device, which in the present embodiment is a haemodiafiltration device. The haemodiafiltration device, which is only described as an example of a blood purification device, has a dialyser (filter) 1 which is separated by a semipermeable membrane 2 into a blood chamber 3 and a dialysis fluid chamber 4. The inlet to the blood chamber 3 is connected by one end to a blood supply line 5, into which a blood pump 6 is connected, while the outlet of the blood chamber is connected by one end to a blood removal line 7, into which a drip chamber 8 is connected. Together with the blood chamber 3 of the dialyser, the blood supply line 5 and blood removal line 7 form the extracorporeal blood circuit 9 of the haemodiafiltration device. The blood supply line 5 and blood removal line 7 are hose lines of a tube set (disposable) inserted into the haemodiafiltration device.

The dialysis fluid system 10 of the haemodiafiltration device comprises an apparatus 11 for providing dialysis fluid, which is connected, by means of the first portion of a dialysis fluid supply line 12, to the inlet of the first balancing chamber half 35a of a balancing apparatus 35. The second portion of the dialysis fluid supply line 12 connects the outlet of the first balancing chamber half 35a to the inlet of the dialysis fluid chamber 4. The outlet of the dialysis fluid chamber 4 is connected to the inlet of the second balancing chamber half 35b by means of the first portion of a dialysis fluid removal line 13. A dialysis fluid pump 14 is connected into the first portion of the dialysis fluid removal line 13. The outlet of the second balancing chamber half 35b is connected to an outflow 15 by means of the second portion of the dialysis fluid removal line 13. An ultrafiltrate line 16, which also leads to the outflow 15, branches off from the dialysis fluid removal line 13 upstream of the dialysis fluid pump 14. An ultrafiltration pump 17 is connected into the ultrafiltrate line 16. In conventional devices, the balancing apparatus 35 consists of two parallel balancing chambers, which can be operated in an anti-cyclical manner.

During the dialysis treatment, the patient's blood flows through the blood chamber 3 and the dialysis fluid flows through the dialysis fluid chamber 4 of the dialyser. By means of the ultrafiltration pump 17, a predetermined amount of fluid (ultrafiltrate) can be removed from the patient at a predetermined ultrafiltration rate. In order to supply fluid back to the patient, the haemodiafiltration device has a substitution apparatus 19, by means of which a substitution fluid (substituate) can be supplied to the blood, which fluid flows through the arterial branch 20 (predilution) and/or the venous branch 21 (postdilution) of the extracorporeal blood circuit 9. The substitution apparatus 19 has an apparatus 37 for providing substituate, from which a first substituate line 36, into which a first substituate pump 22 is connected, leads to the portion of the blood supply line 5 between the blood pump 6 and the blood chamber 3. A second substituate line 23, into which a second substituate pump 24 is connected, leads from the apparatus 37 for providing substituate to the drip chamber 8.

The haemodiafiltration device has a central control and/or computing unit 25 which may have, for example, a general processor, a digital signal processor (DSP) for continuously processing digital signals, a microprocessor, an application-specific integrated circuit (ASIC), an integrated circuit consisting of logic elements (FPGA) or other integrated circuits (IC) or hardware components to perform the individual method steps for controlling the hameodiafiltration device. A data processing program (software) may run on the hardware components in order to carry out the method steps. The data processing program can be stored on a memory of the control and/or computing unit 25.

The central control and/or computing unit 25 is connected to the blood pump 6, the dialysis fluid pump 14, the ultrafiltration pump 17 and the first and second substituate pumps 22, 24 via control lines 6′, 14′, 17′, 22′, 24′. The control and/or computing unit 25 controls the pumps such that the blood purification is performed with a predetermined blood flow rate Qb, dialysis fluid rate Qd and substitution rate Qs.

The device according to the invention for monitoring blood purification is described below as part of the blood purification device. The monitoring device can, however, also be a device that is spatially separated from the blood purification device. However, if the monitoring device is part of the blood purification device, the monitoring device can make use of the components of the blood purification device, in particular the control and/or computing unit 25 thereof.

In the present embodiment, the haemodiafiltration device has a first sensor 31 arranged upstream of the dialysis fluid chamber 4 of the dialyser 1 and a second sensor 32 arranged in the dialysis fluid removal line 16 downstream of the dialysis fluid chamber 4, as well as a third sensor 33 arranged in the blood removal line 7 downstream of the blood chamber 3 and a fourth sensor 34 arranged in the blood supply line 20 upstream of the blood chamber 3, which sensors are designed to measure a variable correlating with the concentration of a substance in the dialysis fluid or the blood.

In the present embodiment, the sensors 31, 32, 33, 34 are conductivity sensors for measuring the conductivity of the blood or the dialysis fluid.

The central computing and/or evaluation unit 25 is configured such that, on the basis of the conductivity measured with at least one of the sensors 31, 32, 33, 34, a parameter is determined which is characteristic of the purifying performance of the dialyser during the blood purification performed with predetermined treatment parameters. In the present embodiment, the conductivity is measured both on the blood side and on the dialysis fluid side. However, not all sensors need to be present to determine this parameter. The computing and/or evaluation unit 25 calculates, during the blood purification, for example, the clearance K according to equation (1) from the measured blood inlet concentration c_{bi} and blood outlet concentration c_{bo} and the blood flow Q_{b}, or the dialysance D according to equation (2) from the blood inlet concentration c_{bi}, blood outlet concentration c_{bo} and the dialysis fluid inlet concentration cdi and the blood flow Q_{b}. In addition, the computing and/or evaluation unit 25 calculates Kt (t: treatment time) or Dt (t: treatment time) and the dialysis dose Kt/V (V: distribution volume) or the dialysis dose Dt/V. However, all other known methods can also be used, for example only on the basis of measurements on the dialysate side.

The computing and/or evaluation unit 25 is configured such that an expected value K_{ref}, which is dependent on at least one treatment parameter, is determined for the purifying performance of the dialyser, with which the parameter determined on the basis of the conductivity measurement, which is characteristic of the purifying performance of the dialyser, is compared. In the present embodiment, the expected value K_{ref} is calculated according to a mathematical model. Such mathematical models are known. In the present embodiment, the expected value is calculated according to the mathematical model described in Sargent “J. A., Gotch. F. A.: Principles and biophysics of dialysis, in: Replacement of Renal Function by Dialysis, W. Drukker, F. M. Parsons, J. F. Maher (ed.). Nijhoff, Den Haag 1983.

$${D}_{diff} = {Q}_{Bi} \frac{{e}^{γ} - 1}{{e}^{γ} - \frac{{Q}_{B}}{{Q}_{d}}} , γ = {k}_{0} A \frac{{Q}_{d} - {Q}_{Bi}}{{Q}_{Bi} {Q}_{d}} \qquad Equation ( 3 )$$

D_{diff} denotes the diffusive portion of the clearance K and Q_{Bi} denotes the entire blood flow at the inlet of the blood chamber 3 of the dialyser 4.

For treatments with HD and HDF post-dilution:

$${Q}_{Bi} = {Q}_{b} ( blood flow ) ; \\ {Q}_{Bi} = Qb + Qs$$

For treatments with HDF predilution:

(substitution rate Qₛ).

The expected value K_{ref} for the clearance K is calculated as follows, taking into account the dialysis method used:

$${K}_{ref} = \frac{{Q}_{b}}{{Q}_{b} + κ {Q}_{s}} ( {D}_{diff} \frac{{Q}_{b} - {Q}_{f} - ( 1 - κ ) {Q}_{s}}{{Q}_{b} + κ {Q}_{s}} + {Q}_{f} + {Q}_{s} ) \\ κ = \{ \begin{matrix}0 & HD , HDF - post \\ 1 & HDF - pre\end{matrix} \qquad Equation ( 4 )$$

For the dialyser parameter K₀A (equation (3)) which describes the properties of the dialyser, use can be made of a value specified by the manufacturer of the dialyser, which can be determined using laboratory measurements.

However, when determining the dialyser parameter K₀A, it has to be taken into account that, for K₀A, an effective value (K₀A)eff would have to be used which deviates substantially from the manufacturer's information derived from laboratory measurements (e.g. Depner “Dialyzer Performance in the HEMO Study: In Vivo K₀A and True Blood Flow Determined from a Model of Cross-Dialyzer Urea Extraction”, ASAIO Journal 2004) and takes into account the actual blood properties and properties of the extracorporeal blood circuit. The computing and/or evaluation unit 25 can therefore also be configured such that, after the determination or measurement of the clearance K, an effective value (K₀A)eff is determined by inverting equation (3) and equation (4) during the blood purification and is then used as the expected value in the same or a later treatment of the same or a different patient to calculate the expected value according to equation (3) and equation (4).

The haemodiafiltration device has a memory unit 38 which, in the present embodiment, is connected to the computing and/or evaluation unit 25 via a data line 39. K₀A or (K₀A)eff can be read into or read out from the memory unit 38 by the computing and/or evaluation unit 25.

The computing and/or evaluation unit 25 is further configured such that a tolerance range is determined for the expected value K_{ref}. The tolerance range is defined by an upper and lower limit value [Kₘᵢₙ, Kₘₐₓ], K_{ref}∈[Kₘᵢₙ, Kₘₐₓ]. The tolerance range can be symmetrical or asymmetrical around K_{ref}. An assumed maximum value can be used as the upper limit for Kₘₐₓ, e.g. for haemodiafiltration (HDF) treatments the smallest value of Q_{bw} (blood water flow) and Qd (dialysis fluid flow), and for haemofiltration (HF) treatments and for absorber treatments less than or equal to Q_{bw}, because the clearance cannot be greater than the flows at the dialyser. The limits of the tolerance range can also be defined on the basis of the deviations from K_{ref} in previous treatments of the same or other patients. For this purpose, the position can be defined based on the standard deviation σ of K_{ref} with Kₘᵢₙ=K_{ref}−Xₘᵢₙσ and Kₘₐₓ=K_{ref}+Xₘₐₓσ. Values for xₘᵢₙ and xₘₐₓ of between 1 and 5 are advantageous here.

In the present embodiment, the computing and/or evaluation unit 25 defines an upper limit value Kₘₐₓ which is a certain percentage, for example 10%, above the expected value K_{ref}, and defines a lower limit value Kₘᵢₙ which is a certain percentage, for example 10%, below the expected value K_{ref}.

The computing and/or evaluation unit 25 is also configured such that it is calculated whether the measured clearance K or dialysance D lies within the tolerance range, i.e. is less than Kₘₐₓ and greater than Kₘᵢₙ. If K or D is greater than Kₘₐₓ or less than Kₘᵢₙ, the computing and/or evaluation unit 25 generates an electrical and acoustic signal that signals that an operating state is present that does not correspond to the ideal or normal operating state.

The haemodiafiltration device has a graphical and acoustic user interface 40 which, in the present embodiment, comprises a touchscreen 40A or a screen and an input device, for example a computer mouse. The computing and/or evaluation unit 25 is connected to the user interface 40 via a data line 41 and interacts with the user interface such that graphical elements and symbols are displayed on the touchscreen 40A that indicate to the user that the parameter which is characteristic of the purifying performance of the blood purification unit lies within or outside the tolerance range for the expected value or that prompt the user to perform certain actions. The user interface has a speaker 40B for outputting acoustic signals, for example an alarm signal.

FIG. 2 shows the screen 40A of the user interface 40. On the screen 40A, the upper limit value Kₘₐₓ is displayed as a horizontal upper line and the lower limit value Kₘᵢₙ is displayed as a horizontal lower line. The tolerance range is the area between the upper and lower limit value. The clearance K or dialysance D measured during the blood purification is displayed as a function of the treatment time t. The measured clearance K or dialysance D can be displayed continuously on the screen or only after the treatment has ended. The user can immediately see on the screen whether the measured clearance K or dialysance D deviates from the expected value by a value that is still tolerable. In FIG. 2, the clearance K is within the tolerance range.

In addition, symbols 42, 43 are displayed on the screen 40A. In the present embodiment, a symbol 42 appears on the screen, for example, which prompts the user to perform a specific action, for example to input specific data. Buttons 44, 45, 46 are also shown on the screen and can be actuated by the user when certain actions are to be carried out. These actions can also be carried out automatically as soon as the computing and/or evaluation unit 25 has determined that the operating state is not normal.

FIG. 3 shows the screen 40A, whereby the clearance K falls below the lower limit value Kₘᵢₙ during the treatment and is thus outside the tolerance range. If the clearance K falls below the lower limit value Kₘᵢₙ, an acoustic alarm signal is generated using the speaker 40B.

In an alternative embodiment, the expected value K_{ref} is not calculated. Expected values K_{ref} for different treatment parameters are stored in the memory 38 in the form of a table. For example, different blood flows Q_{b} are each assigned an expected value. Corresponding tables for different types of dialyser can be stored in the memory 38. The computing and/or evaluation unit 25 is configured such that the relevant expected value K_{ref} for the predetermined treatment parameter, for example the blood flow Qb, or the predetermined treatment parameters, for example blood flow Qb and dialysis fluid flow Qd, is read out from the memory 38 and used as the basis for the further calculation.

The computing and/or evaluation unit 25 can also be configured such that a parameter determined during a prior blood purification with the extracorporeal blood purification device on the basis of a conductivity measurement, which parameter is characteristic of the purifying performance of the dialyser, is input into the memory 38, and this parameter is read out from the memory 38 as the expected value K_{ref} for the blood purification of a subsequent blood purification and used as the basis for the further calculation.

FIG. 4 shows an embodiment which differs from the embodiment described with reference to FIG. 3 in that the memory 38′ is not part of the blood purification device or the device for monitoring blood purification, but is spatially separated from the blood purification device or monitoring device. The blood purification device or monitoring device therefore has a data interface 47 for exchanging data with the memory 38′. The data transfer can take place, for example, via the Internet (cloud computing), and therefore a plurality of blood purification devices can exchange data with one another in order to create a database that can be accessed to read out the appropriate expected value or to read out data for determining the expected value.

FIG. 5 shows a blood purification system which comprises two extracorporeal blood purification devices A and B, for example haemodiafiltration devices, which are described with reference to FIG. 4, and a data processing system C. The blood purification system can also comprise more than two haemodiafiltration devices. The plurality of haemodiafiltration devices A, B interact with the data processing system C such that data are exchanged via their data interface 47 between the plurality of haemodiafiltration devices on the one hand and/or between a haemodiafiltration device and the data processing system on the other hand. The control and/or computing units 25 of the haemodiafiltration devices A, B and/or the data processing system C are configured such that, for example, the clearance K or dialysance D is read into a memory as a parameter which is characteristic of the purifying performance of the dialyser and which is determined during blood purification with one of the haemodiafiltration devices on the basis of a conductivity measurement, and is read out from the memory by a different haemodiafiltration device as the expected value. The memory can be a memory 38 of the haemodiafiltration device A, B and/or a memory 38″ of the data processing system C. The data transmission can take place, for example, via the Internet (cloud computing).

The parameter which is characteristic of the purifying performance of the blood purification unit and is determined on the basis of a conductivity measurement can also be (K₀A)eff. After the measurement of K or D (equation (1) or equation (2)) in a prior blood purification, (K₀A)eff can be calculated according to equation (3) and (4) and read into the memory and (K₀A)eff can be read out from the memory as the expected value for the blood purification of a subsequent blood purification and used as the basis for the further calculation.

Since the clearance K clearly denotes the proportion of the blood flow that has been completely freed of the substance of interest, a comparison of the clearance K with the blood flow Q_{b} or a comparison of Kt (t: treatment time) with the blood distribution volume V_{b} is particularly informative.

This results in the following reference variables:

$$Kref = frefQb \\ ( Kt ) ref = frefvb \qquad Equation ( 5 )$$

fref can be determined on the basis of theoretical considerations or information from the manufacturer of the blood purification unit, or on the basis of measurements during the current treatment of the patient or measurements during prior treatments of the same or other patients. In this case, it is advantageous to use only the blood water flow Q_{bw} as a reference instead of the entire whole blood flow Q_{b}.

$${f}_{ref}^{′} = {f}_{ref} {f}_{bw} \qquad Equation ( 6 )$$

Formulas are known in the literature for the determination of f_{bw} from haematocrit and plasma protein fraction. A typical value is f_{bw}=0.86.

Application examples of the method according to the invention and the blood purification device according to the invention are described below.

In the case of an extracorporeal blood purification, the problem arises that recirculation occurs in the vascular access if the blood flow at the vascular access falls below the extracorporeal blood flow, and therefore the purifying performance is reduced. This can be detected on the basis of a decrease in the clearance K below a historically determined reference value. This is explained below using a real clinical example.

During a patient's treatment, the parameters Q_{b}, Q_{d}, Qₛ, K, V_{b} and Kt were registered and evaluated over a period of approximately 6 months. Various methods were used to detect a deviation in the clearance from the normal operating state. A sliding mean value was continuously formed over these parameters or over derived parameters and a tolerance range was determined from the variation (standard deviation σ), with a width of ±4σ around the mean value being used in the example shown. After falling below the tolerance range, the previously valid tolerance range was no longer updated.

FIGS. 6A, 6B and 6C show the result of the analysis based on individual measurements of the clearance, which were carried out several times per treatment. The following references were used to compare the measured parameter which is characteristic of purifying performance.

FIG. 6A Blood flow Qb

FIG. 6B Reference clearance K_{ref}, which was calculated from Qb, Qd, Qf and Qs assuming a fixed K₀A (=460 ml/min).

FIG. 6C Fixed K₀A=460 ml/min. The current effective value of K₀A was then calculated from the measured clearance and Qb, Qd, Qf and Qs by inverting equation (5) and (6).

To assess the selectivity of the method, the signal to noise ratio (S/N) was determined, which is defined as the amplitude between the mean value of the reference period and the minimum of the parameter, divided by the standard deviation in the reference period (FIG. 6A S/N=9.7; FIG. 6B S/N=12.5; FIG. 6C S/N=5.9)

All analyses show a fall below the tolerance range at the start of July 2013, with a low point in the middle of July 2013. Thereafter, according to clinical reports, a revision of the vascular access took place, which corrected the problem. The best S/N was achieved using the reference clearance K_{ref} calculated from the clearance model described above (equations (3) and (4)).

FIG. 7A, 7B, 7C show trend analyses for K_{ref}/Q_{b} (FIG. 7A), K_{ref}/K_{ref model} (FIG. 7B), K₀A/K₀Astd (FIG. 7C) based in each case on only one clearance measurement in the middle of the treatment. All analyses showed an improved S/N [(FIG. 7A: S/N=16.4), (FIG. 7B: S/N=16.5), (FIG. 7C: S/N=8.1). This is due to the fact that other effects during dialysis influence the course of clearance, and therefore better reproducibility can be achieved in the middle of the treatment at comparable time points.

FIG. 8 shows an analysis in which the blood water flow Q_{bw} was used for normalization instead of the whole blood flow Q_{b}. The S/N remains unchanged.

FIGS. 9A, 9B and 9C show trend analyses for K_{ref}*t/V_{b} (time integral instead of current value) (FIG. 9A), K_{ref}*t/K_{ref} model*t (FIG. 9B), K0A/K0Astd (FIG. 9C) based on the total dialysis dose achieved in the respective treatments, which results from the integration of the individual clearance values of a treatment. To calculate K₀A, mean values were formed from the individual clearance values and the flows. All analyses show an improved S/N compared to the analysis based on individual clearance values, with the best selectivity in turn being able to be achieved on the basis of a comparison of the measured clearance with the clearance model as reference.

It is apparent that all the methods described are suitable for detecting a problem in the vascular access. The use of a mathematical model for determining the reference clearance based on historical data is advantageous here, and the use of parameters averaged over the course of the treatments is particularly advantageous compared to the use of the data from individual measurements.

In the case of blood purification, there is also the problem that the actual delivery rate of the blood pump can deviate from the expected value. This deviation can have various causes. With peristaltic pumps, an arterial negative pressure and the softening of the pump hose segment can lead to a reduction in the delivery rate at constant speed. With impeller pumps, the delivery rate is primarily influenced by the flow resistance in the dialyser, and therefore in extreme cases there is no flow despite the rapidly rotating pump. This can also occur with peristaltic pumps if the hose system is kinked in front of the dialyser. As a consequence of the effectively reduced blood flow, the clearance decreases.

If the clearance is measured with conductivity measurements on the dialysate side, a deviation from the normal or ideal operating state can be detected on the basis of a comparison of the measured clearance with an expected value of the clearance, which can be calculated using the predetermined blood purification parameters.

The course of the concentration of a certain substance or substance class can be measured during a dialysis treatment with suitable sensors downstream of the dialyser. Such sensors can be based on the measurement of the absorption in the infrared or visible range of light or in the UV range of light. Alternatively, the fluorescent light can also be determined when excited at a preferred wavelength (approx. 250-450 nm). It is also possible to use Raman spectroscopy. Alternatively, substance-specific chemosensors are also possible. The fractional substance-specific dialysis dose Kt/V can then be calculated from a signal proportional to the concentration profile. If the substance-specific distribution volume is known, the substance-specific clearance K can also be calculated. This substance-specific clearance can then also be compared with corresponding reference values using the methods described.

Furthermore, it is advantageous, when simultaneously determining the low-molecular-weight dialyser clearance, to normalise the substance-specific clearance to the low-molecular-weight dialyser clearance and to compare it with corresponding reference values using the known methods. If the substance under consideration is, for example, a middle molecule that is mainly removed by convection, a decrease in the substance-specific or normalised substance-specific clearance would indicate an error in the administration of the substitution solution (e.g. technical error in the substitution pump, kinking of the substitution hose). Alternatively, a possible cause could be clogging of the pores of the dialyser. An increased substance-specific clearance, e.g. in the case of albumin, could indicate the use of a membrane that is too open-pored (e.g. use of a medium cutoff filter for HDF, batch problem, etc.).

1. A method for monitoring blood purification with an extracorporeal blood purification device which is designed such that blood purification is carried out with predetermined treatment parameters by means of a blood purification unit in an extracorporeal blood circuit, wherein the concentration of a substance or a variable correlating with the concentration of a substance is measured with at least one sensor during the blood purification and, on the basis of the concentration of a substance measured with the at least one sensor or a variable correlating with the concentration of a substance, at least one parameter which is characteristic of the purifying performance of the blood purification unit during the blood purification with the predetermined treatment parameters is determined using a computing and/or evaluation unit, wherein the extracorporeal blood purification device is an extracorporeal haemodialysis device, haemofiltration device or haemo (dia) filtration device, the blood purification unit of which has a first compartment and a second compartment which are separated by a semipermeable membrane, wherein the first compartment is part of an extracorporeal blood circuit and the second compartment is part of a dialysis fluid system, and the at least one sensor is provided for measuring the concentration of a substance or a variable correlating with the concentration of a substance in the extracorporeal blood circuit and/or in the dialysis fluid system, and the parameter which is characteristic of the purifying performance of the blood purification unit is the clearance and/or dialysance and/or the dialysis parameter of the dialysis treatment, and wherein
an expected value for the purifying performance of the purification unit, which value is dependent on at least one treatment parameter, is determined using the computing and/or evaluation unit, and in that a tolerance range is determined for the expected value using the computing and/or evaluation unit, wherein actions that are predetermined by the computing and/or evaluation unit are triggered depending on whether the parameter which is characteristic of the purifying performance of the blood purification unit lies within or outside the tolerance range for the expected value, a predetermined treatment parameter being the blood flow.

2. The method for monitoring blood purification according to claim 1, wherein graphical elements and/or symbols are displayed with a graphical user interface and/or acoustic signals are generated with an acoustic user interface, which interfaces display to the user that the parameter which is characteristic of the purifying performance of the blood purification unit lies within or outside the tolerance range for the expected value.

3. The method for monitoring blood purification according to claim 1 in that wherein an electrical signal is generated which signals that the parameter which is characteristic of the purifying performance of the blood purification unit is within or outside the tolerance range for the expected value.

4. The method for monitoring blood purification according to claim 1, wherein expected values for different treatment parameters are stored in a memory, wherein the relevant expected value for the predetermined treatment parameters is read out from the memory by the computing and/or evaluation unit.

5. The method for monitoring blood purification according to claim 1, wherein the computing and/or evaluation unit is used to calculate the expected value according to a mathematical model which describes the expected value as a function of the predetermined treatment parameters.

6. The method for monitoring blood purification according to claim 1, wherein a parameter which is characteristic of the purifying performance of the blood purification unit is determined during a prior blood purification with the extracorporeal blood purification device and is stored in a memory, wherein the expected value determined during the prior blood purification with the extracorporeal blood purification device is read out from the memory as the expected value for the blood purification of a subsequent blood purification.

7. The method for monitoring blood purification according to claim 1, wherein a parameter is determined which is characteristic of the purifying performance of the blood purification unit during blood purification with a different extracorporeal blood purification device than the one to be monitored and is stored in a memory, wherein the expected value determined during a prior blood purification with the different extracorporeal blood purification device is read out from the memory as the expected value for the blood purification with the blood purification device to be monitored.

8. The method for monitoring blood purification according to claim 1, wherein the computing and/or evaluation unit is used to calculate the expected value according to a mathematical model which describes the clearance K and/or dialysance D and/or the dialysis parameter K₀A as a function of predetermined treatment parameters.

9. The method for monitoring blood purification according to claim 1, wherein the computing and/or evaluation unit is a computing and/or evaluation unit which is spatially separated from the blood purification device.

10. The method for monitoring blood purification according to claim 4, wherein the memory is a memory which is spatially separated from the blood purification device.

11. A device for monitoring blood purification for use with an extracorporeal blood purification device which is designed such that a blood purification unit is used to perform blood purification with predetermined treatment parameters in an extracorporeal blood circuit, wherein the device for monitoring blood purification has at least one sensor for measuring the concentration of a substance or a variable correlating with the concentration of a substance during the blood purification and a computing and/or evaluation unit which is configured such that, on the basis of the concentration of a substance measured with the at least one sensor or a variable correlating with the concentration of a substance, at least one parameter is determined which is characteristic of the purifying performance of the blood purification unit during the blood purification with the predetermined treatment parameters, wherein the extracorporeal blood purification device is an extracorporeal haemodialysis device, haemofiltration device or haemo(dia)filtration device, the blood purification unit of which has a first compartment and a second compartment which are separated by a semipermeable membrane, wherein the first compartment is part of an extracorporeal blood circuit and the second compartment is part of a dialysis fluid system, and the at least one sensor is provided for measuring the concentration of a substance or a variable correlating with the concentration of a substance in the extracorporeal blood circuit and/or in the dialysis fluid system, and the parameter which is characteristic of the purifying performance of the blood purification unit is the clearance and/or dialysance and/or the dialysis parameter of the dialysis treatment, wherein
the computing and/or evaluation unit is configured such that an expected value is determined for the purifying performance of the blood purification unit, which value is dependent on at least one treatment parameter, and in that a tolerance range is determined for the expected value, wherein predetermined actions are triggered depending on whether the parameter which is characteristic of the purifying performance of the blood purification unit lies within or outside the tolerance range for the expected value, a predetermined treatment parameter being the blood flow.

12. The device for monitoring blood purification according to claim 11, wherein the computing and/or evaluation unit interacts with a graphical user interface which is designed such that graphical elements and/or symbols are displayed, and/or interacts with an acoustic user interface that is designed to generate acoustic signals that indicate to the user that the parameter which is characteristic of the purifying performance of the blood purification unit is within or outside the tolerance range for the expected value.

13. The device for monitoring blood purification according to claim 11, wherein the computing and/or evaluation unit is configured such that an electrical signal is generated which signals that the parameter which is characteristic of the purifying performance of the blood purification unit is within or outside the tolerance range for the expected value.

14. An extracorporeal blood purification device which is designed such that a blood purification unit is used to perform blood purification with predetermined treatment parameters in an extracorporeal blood circuit, wherein the extracorporeal blood purification device has a device for monitoring the blood purification according to claim 1.

15. The extracorporeal blood purification device according to claim 11, wherein the computing and/or evaluation unit is designed such that the relevant expected value for the predetermined treatment parameters is read out from a memory of the blood purification device, in which memory expected values for different treatment parameters are stored.

16. The extracorporeal blood purification device according to claim 11, wherein the computing and/or evaluation unit is configured such that the expected value is calculated according to a mathematical model which describes the expected value as a function of the predetermined treatment parameters.

17. The extracorporeal blood purification device according to claim 11, wherein the computing and/or evaluation unit is configured such that a parameter which is characteristic of the purifying performance of the blood purification unit and determined during a prior blood purification with the extracorporeal blood purification device is stored in a memory of the blood purification device, and the expected value determined during the prior blood purification with the extracorporeal blood purification device is read out from the memory as the expected value for the blood purification of a subsequent blood purification.

18. The extracorporeal blood purification device according to claim 11, wherein the computing and/or evaluation unit is configured such that the expected value is calculated according to a mathematical model which describes the clearance and/or dialysance and/or the dialysis parameter of the dialysis treatment as a function of predetermined treatment parameters.

19. A blood purification system comprising at least two blood purification devices, each designed such that blood purification with predetermined treatment parameters is performed by means of a blood purification unit in an extracorporeal blood circuit, wherein the blood purification devices each have a data interface, and comprising a data processing system with which the at least two blood purification devices interact such that data are exchanged via the data interface between the at least two blood purification devices on the one hand and/or between at least one of the blood purification devices and the data processing system, on the other hand, wherein
the blood purification devices each have at least one sensor for determining the concentration of a substance or a variable correlating with the concentration of a substance during the blood purification and a computing and/or evaluation unit which is configured such that, on the basis of the concentration of a substance measured with the at least one sensor or a variable correlating with the concentration of a substance, at least one parameter is determined which is characteristic of the purifying performance of the blood purification unit during the blood purification with the predetermined treatment parameters, and
the blood purification system comprises a computing and/or control unit which is configured such that a parameter which is characteristic of the purifying performance of the blood purification unit, which parameter is determined during a blood purification with one of the at least two blood purification devices, is read into a memory and is read out from the memory by another blood purification device of the at least two blood purification devices as the expected value, wherein the extracorporeal blood purification device is an extracorporeal haemodialysis device, haemofiltration device or haemo(dia)filtration device, the blood purification unit of which has a first compartment and a second compartment which are separated by a semipermeable membrane, wherein the first compartment is part of an extracorporeal blood circuit and the second compartment is part of a dialysis fluid system, and the at least one sensor is provided for measuring the concentration of a substance or a variable correlating with the concentration of a substance in the extracorporeal blood circuit and/or in the dialysis fluid system, and the parameter which is characteristic of the purifying performance of the blood purification unit is the clearance and/or dialysance and/or the dialysis parameter of the dialysis treatment, a predetermined treatment parameter being the blood flow.
